(19) United States
(12) Patent Application Publication
(10) Pub. No.: US 2010/0081186 A1
(43) Pub. Date: Apr. 1, 2010

(54) Self-decontaminating metal organic frameworks

(76) Inventors: Yongwoo Lee, Watham, MA (US); Tomasz Modzelewski, Lawrenceville, NJ (US); John P. Puglia, Townsend, MA (US); Steven E. Weiss, Auburndale, MA (US); Banglin Chen, San Antonio, TX (US)

Correspondence Address: Iandiorio Teska & Coleman, 260 Bear Hill Road, Waltham, MA, 02451, US

(21) Appl. No.: 12/584,601

(22) Filed: Sep. 9, 2009

Related U.S. Application Data
(60) Provisional application No. 61/194,769, filed on Sep. 30, 2008.

(51) Int. Cl. A62D 3/02; A62D 3/36; C07F 1/08; C12N 11/02

(52) U.S. Cl. 435/177; 588/318; 435/262.5; 544/225

(57) ABSTRACT

A self-decontaminating metal organic framework including an acid linked to a metal producing a metal organic framework configured for the sorption of chemical warfare agents and/or toxic industrial chemicals, the metal organic framework including reactive sites for the degradation of the agents and chemicals.

### RELATED APPLICATIONS

This application hereby claims the benefit of and priority to U.S. Provisional Application Ser. No. 61/194,769, filed on Sep. 30, 2008 under 35 U.S.C. §§119, 120, 363, 365, and 37 C.F.R. §1.55 and §1.78, which is hereby incorporated herein by reference.

### FIELD OF THE INVENTION

This invention relates to protection against chemical warfare agents and toxic industrial chemicals.

### BACKGROUND OF THE INVENTION

Chemical warfare agents (CWAs) and toxic industrial chemicals (TICs) pose a severe human hazard.

In the prior art, carbon may be used in protective clothing, in filters, and the like. Activated carbon is a very good adsorbent of CWAs and TICs. One problem is that the carbon itself becomes contaminated.

Carbon-based systems are also quickly saturated since the carbon also absorbs relatively harmless chemicals such as exhaust gases and the like. Protective clothing including carbon is also heavy, cumbersome, and hot. See, e.g., U.S. Pat. No. 6,792,625, incorporated by reference herein.

Several metal-organic framework (MOF) materials are known and have been studied because of their porous nature. It has been suggested to use MOF materials for hydrogen storage. See, e.g., U.S. Pat. Nos. 6,929,679, and 7,343,747, both incorporated by reference herein. See also Chen, Ockwig, Millward, Contreras, and Yaghi, High H₂ Absorption in Microporous Metal-Organic Framework with Open Metal Sites, Angew. Chem. Int. Ed. (2005) pp: 4735-4749 (disclosing MOF-505), incorporated by reference herein.

MOF materials, due to their high and permanent porosity, offer a potential substitute for carbon-based systems used in protective clothing and filters to protect people against CWAs and TICs.

The result in clothing, for example, would not become saturated as quickly, would be less heavy and cumbersome, and not as hot. But, known MOF materials do not chemically degrade CWA and TIC compounds.

### BRIEF SUMMARY OF THE INVENTION

It is therefore an object of this invention to provide new MOFs.

It is a further object of this invention to provide such MOFs which are self-decontaminating.

It is a further object of this invention to provide such self-decontaminating MOFs which can be used to protect people from CWAs and TICs.

This invention features a self-decontaminating metal organic framework which includes an acid linked to a metal producing a metal organic framework configured for the sorption of chemical warfare agents and/or toxic industrial chemicals. The metal organic framework includes reactive sites for the degradation of said agents and chemicals.

In one embodiment, the acid may be a triple bonded acid. The acid may be acetylenedicarboxylic acid (ADA). The metal may be copper nitrate. The self-decontaminating metal organic framework may be linked to the metal with a linking agent. The linking agent may include Pyrazine, 2,6-dimethylpyrazine, 2-6-dichloropyrazine, dipyridylethlene, 4,4′-dipyridyl, or 2,3,5,6-tetramethylpyrazine. The enzyme added to the metal organic framework to may assist in the degradation of said agents and chemicals. The non-self-decontaminating metal organic framework may be added to the self-decontaminating metal organic framework. The size of the pores of the self-decontaminating metal organic framework may be tailored for specific said agents and chemicals. The surface area of the self-decontaminating metal organic framework may be tailored for specific said agents and chemicals.

This invention also features a method for producing a self-decontaminating metal organic framework, the method including combining an acid with a linking agent and a metal to produce a self-decontaminating metal organic framework for sorption of chemical warfare agents and/or toxic industrial chemicals. The self-decontaminating metal organic framework may include reactive sites for the degradation of said agents and chemicals.

In another embodiment, the acid may be a triple bonded acid. The acid may be acetylenedicareoxylic acid (ADA). The metal may be copper nitrate. The linking agent may include Pyrazine, 2,6-dimethylpyrazine, 2-6-dichloropyrazine, dipyridylethlene, 4,4′-dipyridyl, or 2,3,5,6-tetramethylpyrazine. The method may include the step of adding an enzyme to the metal organic framework to assist in the degradation of said agents and chemical. The size of the pores of the self-decontaminating metal organic framework may be tailored for specific said agents and chemicals. The surface area of the self-decontaminating metal organic framework may be tailored for specific said agents and chemicals.

This invention further features a method of absorbing and degrading chemical warfare agents and toxic industrial chemicals, the method including adding a self-decontaminating metal organic framework to fabric or filter material, the self-decontaminating metal organic framework comprising an acid linked to a metal-organic framework for the sorption of chemical warfare agents and/or toxic industrial chemicals. The metal organic framework may include reactive sites for the degradation of said agents and chemicals.

In another embodiment, the acid may be a triple bonded acid. The acid may be acetylenedicareoxylic acid. The metal may be copper nitrate. The self-decontaminating metal organic framework may be linked to the metal with a linking agent. The linking agent may include Pyrazine, 2,6-dimethylpyrazine, 2-6-dichloropyrazine, dipyridylethlene, 4,4′-dipyridyl, or 2,3,5,6-tetramethylpyrazine. The method may include an enzyme added to the metal organic framework to assist in the degradation of said agents and chemicals. The size of the pores of the self-decontaminating metal organic framework may be tailored for specific said agents and chemicals. The surface area of the self-decontaminating metal organic framework may be tailored for specific said agents and chemicals.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1A shows one combination of an acid, a linking agent, and a metal combined to produce one embodiment of the self-decontaminating metal organic framework (SD-MOF) of this invention;

FIG. 1B shows another combination of an acid, a linking agent and a metal combined to produce another embodiment of the SD-MOF of this invention;

FIG. 1C shows the same combination of an acid, linking agent and metal compound shown in FIG. 1B wherein a different solvent is utilized to produce yet another embodiment of the SD-MOF of this invention;

FIG. 1D shows another combination of an acid, a linking agent and a metal combined to produce another embodiment of the SD-MOF of this invention;

FIG. 1E shows another combination of an acid, linking agent and metal combined to produce another embodiment of the SD-MOF of this invention;

FIG. 1F shows yet another combination of an acid, linking agent and metal combined to produce yet another embodiment of the SD-MOF of this invention;

FIG. 2 shows the chemical structure of various linking agents used to create the SD-MOF of this invention;

FIG. 3 is a three-dimensional view exemplifying the reactive sites of the SD-MOF of this invention;

FIG. 4 shows one example of a self-decontamination reaction of a CWA stimulant which occurs at the reaction sites shown in FIG. 3;

FIG. 5 shows the visual observations of the decomposition of a CWA stimulant using one embodiment of the SD-MOF of this invention;

FIG. 6 is a bar chart showing the SD-MOF of this invention containing and decontaminating a CWA;

FIG. 7 is a graph showing the SD-MOF of this invention to decontaminating a CWA;

FIG. 8 is a bar graph showing one example of the SD-MOF of this invention being reused several times to decontaminate CWAs;

FIG. 9 is a graph showing the activity of enzyme supported reactive adsorbents on the SD-MOF of this invention;

FIG. 10 shows one example of a packed bed reactor (PBR) used to test the decontamination activity of the SD-MOF of this invention; and

FIGS. 11A and 11B are graphs showing the breakthrough of the breakdown product in the PBR shown in FIG. 10;

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

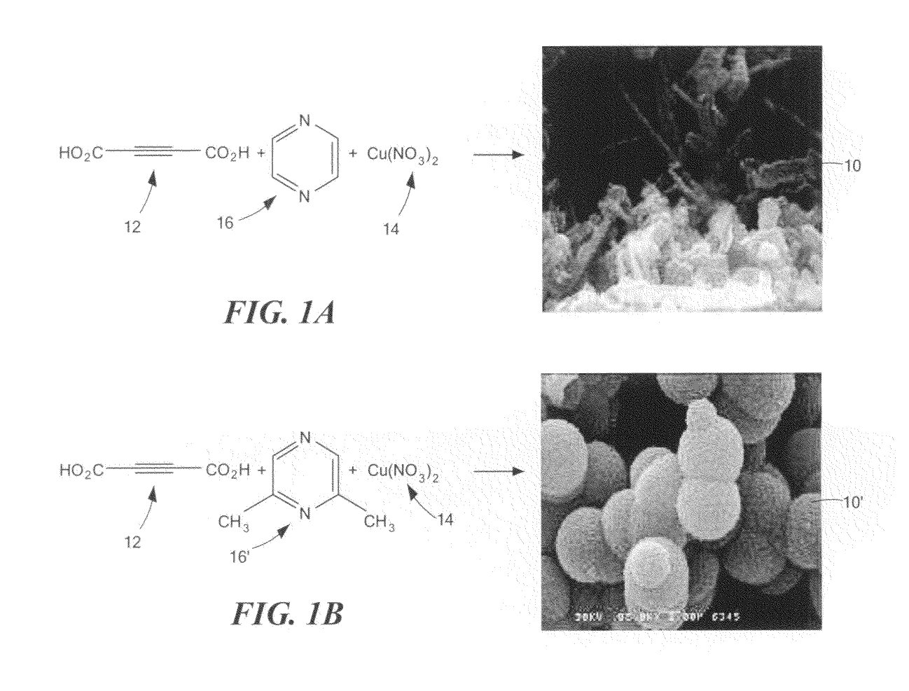

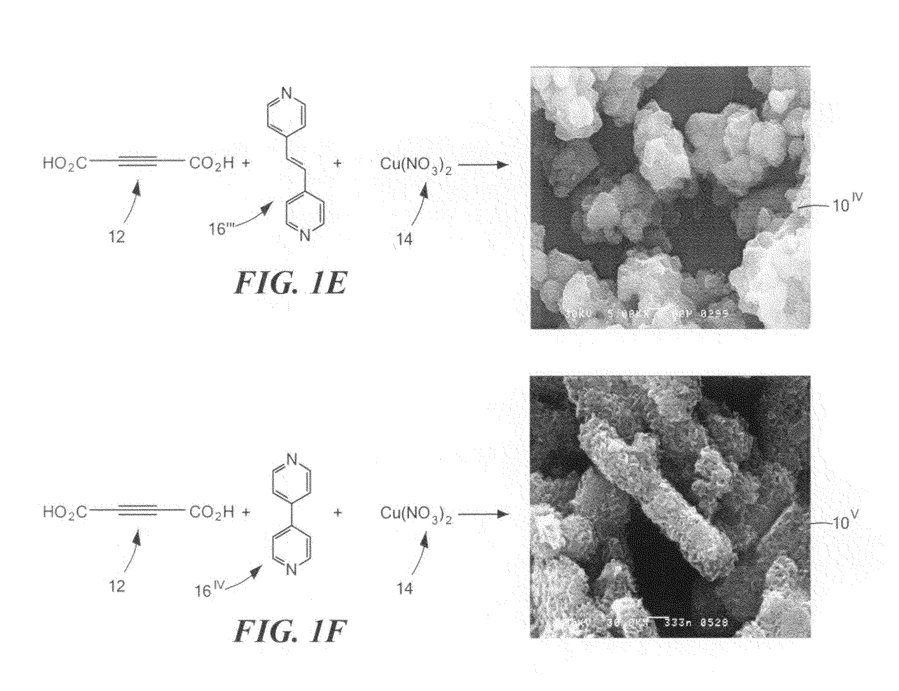

There is shown in FIG. 1A one embodiment of self-decontaminating metal organic framework (SD-MOF) 10 of this invention. SD-MOF 10 is produced by combining acid 12 with metal 14. Preferably, acid 12 is a triple bonded acid, as shown, such as acetylenedicarboxylic acid (ADA), and metal 14 is copper nitrate Cu(NO₃)₂. Other equivalent triple bonded acids and metals may be utilized, as known by those skilled in the arts. Preferably, linking agent 16 is used to combine acid 12 with metal 14, e.g., via a chelating reaction in a solvent. In this example, linking agent 16 is Pyrazine (Pyz) and the solvent is a 1:1:1 mixture of N,N'-dimethyl formamide (DMF):methanol:water at 65° C. SD-MOF 10 is configured for the sorption of chemical warfare agents and/or toxic industrial chemicals and includes reactive sites 20, FIG. 3, (discussed below) which degrade the chemical warfare agents (CWAs) and/or toxic industrial chemicals (TICs).

SD-MOF 10′, FIG. 1B, may be similarly produced by combining acid 12 and metal 14 with a different linking agent 16′, namely, 2,6-dimethylpyrazine. In this example the solvent is water at 90° C.

SD-MOF 10″, FIG. 1C, may be produced by combining the same acid 12, the same metal 14 and the same linking agent 16′ as shown in FIG. 1B with a different solvent:a 1:1:1 mixture of N,N'-dimethyl formamide (DMF):methanol:water at 65° C.

SD-MOF 10′″, FIG. 1D, is produced by combining acid 12 and the metal 14 with yet another different linking agent 16″, namely, 2,6-dichloropyrazine and a solvent of water at 90° C.

SD-MOF 10^{IV}, FIG. 1E, may be produced by combining acid 12 and metal 14 with yet another linking agent 16′″: dipyridylethylene (trans-1,2-bis(4-pyridy)-ethylene) (DPe). In this example the solvent is a 1:1:1 mixture of DMF:methanol:water at 65° C.

In yet another design, SD-MOF 10^{v}, FIG. 1F, is produced by combining acid 12 and metal 14 with yet another linking agent 16^{iv}: 4,4′-dipyridyl (Dpl).

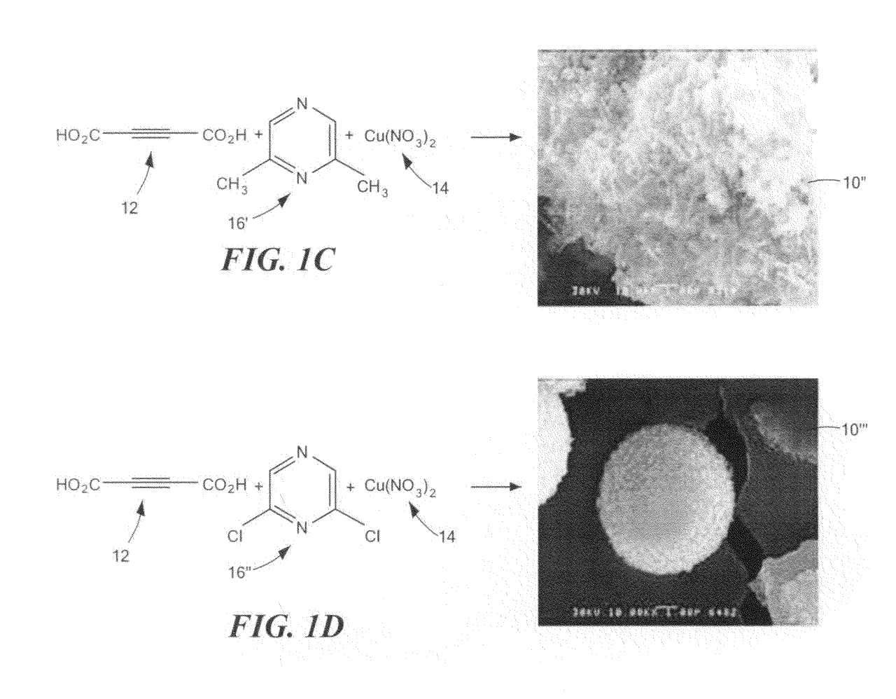

FIG. 2 shows in further detail the chemical structure of linking agent 16, FIG. 1A, linking agent 16′, FIGS. 1B-1C, linking agent 16″, FIG. 1D, and linking agent 16′″, FIG. 1E, which may be used to link acid 12 to metal 14 to yield SD-MOF 10 of this invention. Linking agent 16 may also include other derivatives thereof as known to those skilled in the art.

SD-MOF 10, FIGS. 1A-1F, of this invention includes reactive sites 20, FIG. 3, which degrade CWAs, and/or TICs, e.g., CWAs-22. Because SD-MOF 10 is highly porous, it provides for sorption (adsorption and/or absorption) of CWAs and/or TICs Once adsorbed or absorbed to SD-MOF 10, the CWAs and/or TICs react with reactive sites 20, e.g. a reactive amine or similar type compound, and undergo a chemical reaction which degrades them. For example, CWAs 22 are shown adsorbed to SD-MOF 10 at 24. CWAs 22 then react with reaction sites 22, e.g., as shown at 26, and undergo chemical reactions (discussed below) which degrades the CWAs-22 into non-toxic (NT) chemicals 28.

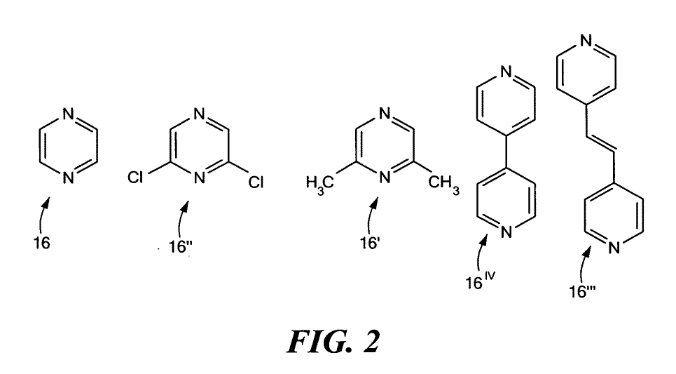

For example, one known simulant of a CWA is methyl parathion (MPT) 30, FIG. 4. When exposed to SD-MOF 10, FIGS. 1A-1F, of this invention, the pores in SD-MOF 10 provide for the sorption of MPT 30. MPT 30, FIG. 4, then reacts with reactive sites 20, FIG. 3, and undergoes the hydrolysis reaction as shown in FIG. 4 to yield non-lethal CWAs, p-Nitrophenol (pNP) 32 and methylthyophosphenic acid 34. The result is SD-MOF 10 has effectively degraded or decontaminated the toxic CWA simulant MPT 30.

Preferably, SD-MOF 10 of this invention is added to a fabric or filter material which may be used as protective clothing and/or filters and the like, to protect people from CWAs and TICs. Because SD-MOF 10 is self-decontaminating and reactive with CWAs and TICs, any protective clothing or filters made from it does not need to be replaced after one use. The protective clothing made from the SD-MOF of this invention is also lighter and less cumbersome than conventional protective clothing made with carbon or similar type materials.

In one embodiment, an enzyme, such as organophosphorous hydrolase (OPH) may be added to SD-MOF to assist in the degradation of CWAs or TICs. Other enzymes known to those skilled in the art may be utilized.

Non self-decontaminating metal organic frameworks may be added to SD-MOF 10 to further increase its porosity. The size of the pores of SD-MOF 10 may be tailored for specific CWAs and TICs, e.g., in the range of about 4 Å to about 12 Å. Similarly, the surface area of SD-MOF 10 may be tailored for specific CWAs and TICs. In one example, SD-MOF 10^{V}, FIG. 1F, has a surface area of about 122 m²/g. Other pore sizes and surface areas may be used as known by those skilled in the art.

### EXAMPLES

The following examples are meant to illustrate and not limit the present invention.

Amine-based linker chemistries may be used to create SD-MOF 10 of this invention. This may be accomplished by combining pyridinyl amine linkers with linear acetylenedicarboxylic acid (ADA) and hydrothermally treating these chemicals in the presence of copper cations at 90-100° C. Examples of active pyridinyl amine linkers, or linking agents 16, are discussed above with reference to FIGS. 1A-1F and FIG. 2. The resulting SD-MOFs may have a Cu:Pyridyl amine molar ratio that approaches about 1:1.

Linking agents 16 can be utilized to alter adsorbent selectivity and activity of SD-MOF 10. SD-MOF 10 may be created though a chelating reaction in either water or a 1:1:1 mixture of N,N′-dimethyl formamide (DMF):methanol:water. Both techniques create a final SD-MOF 10 that shows activity against CWAs and TICs. Reactivity has been observed for both a liquid environment (e.g. a solution of MPT and MPO) and a gas environment (e.g. flowing a stream of nitrogen spiked with MPO vapors at ambient condition). Examples of the various embodiments of the SD-MOF of this invention are shown in FIGS. 1A-1F. The chemical linkers, linking agents 16, are also shown in FIGS. 1A-1F and FIG. 2. The ratio of carboxylic acid to amine functionalized linker is typically about 1:1.

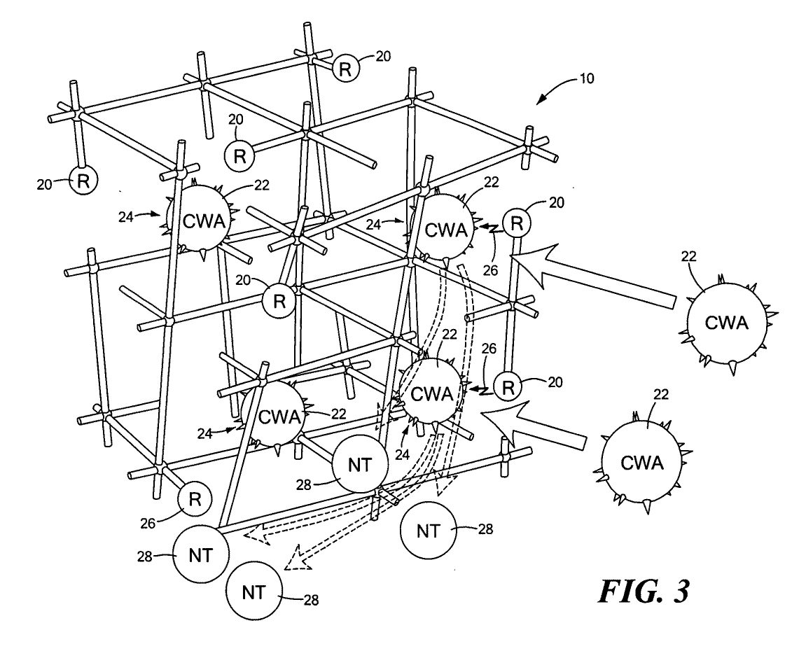

In one example, the chemical reactivity of one or more of SD-MOF 10, FIGS. 1A-1F, hereinafter SD-MOF, was observed towards degradation of MPT simulant. A concentrated yellow-green color rapidly developed in the reaction mixture indicating the appearance of p-nitrophenol (pNP) as a result of decontamination. Reaction progress was monitored via UV-VIS, e.g., disappearance of MPT at 275 nm and the appearance of pNP at 405 nm. Visual observations are shown in FIG. 5. The reaction was reproduced several times with no observable loss in the quantity of the SD-MOF indicating at a minimum a large capacity towards this reaction. As shown 50, FIG. 5, the SD-MOF of this invention is crystalline and contains high Cu:Amine molar content. Room temperature decomposition of the MPT simulant, was demonstrated over the SD-MOF by producing a yellow-green decomposition product, pNP, shown at 52.

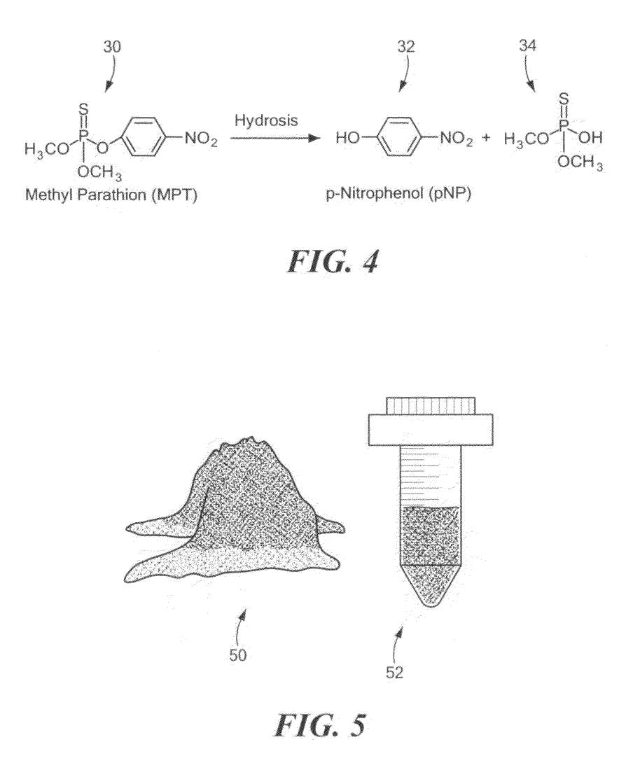

The chemical activity of the SD-MOF of this invention towards MPT hydrolysis was observed using UV-VIS. The appearance of pNP was monitored immediately when 100 μmolar MPT solution was exposed to 100 mg of SD-MOF. It was noticed that the amount of pNP was less than 100% conversion, indicating the partial sorption of MPT to SD-MOF powders during decontamination. To this solution, NaOH was added with no additional pNP production observed. Thus it was concluded that the solution had no residual MPT present in the bulk solution. Therefore, a 100% conversion was indicated. Next, the particles were collected from solution and washed with either DMF or acetone. Additional pNP was collected indicating that the missing pNP was actually present but adsorbed in the MOF structure. Graphs 54 and 60, FIG. 6, show a control NaOH solution exposed to MPT where approximately 100% of the MPT toxic is degraded to non-toxic pNP by products. Graph 56 shows about 85% of the MPT was degraded to pNP in solution (bulk solution) and graph 58 shows about 17% of the MPT was degraded and then absorbed to the particles of the SO-MOF after the reaction was complete and the SD-MOF was rinsed with DMF. Similarly graph 62 shows about 65% of the MPT was degraded to pNP in bulk solution and graph 64 shows about 18% of the MPT was degraded to the SD-MOF particles after the reaction was complete and rinsed with acetone. The above shows the SD-MOF particle is able to decontaminate the MPT from a 15% methanol aqueous solution. The difference between the observed pNP concentration in the bulk solution (graphs 56 and 62) and what is retrieved from the same 100 μM MPT solution, treated with NaOH, (graphs 54 and 60) can be recovered from the SD-MOF particles using DMF or Acetone rinses. MPT was not found in SD-MOF powders when rinsed, but its degraded pNP was observed in the powders as adsorbed (graphs 58 and 64). This indicates complete decontamination by the action of the SD-MOF of this invention.

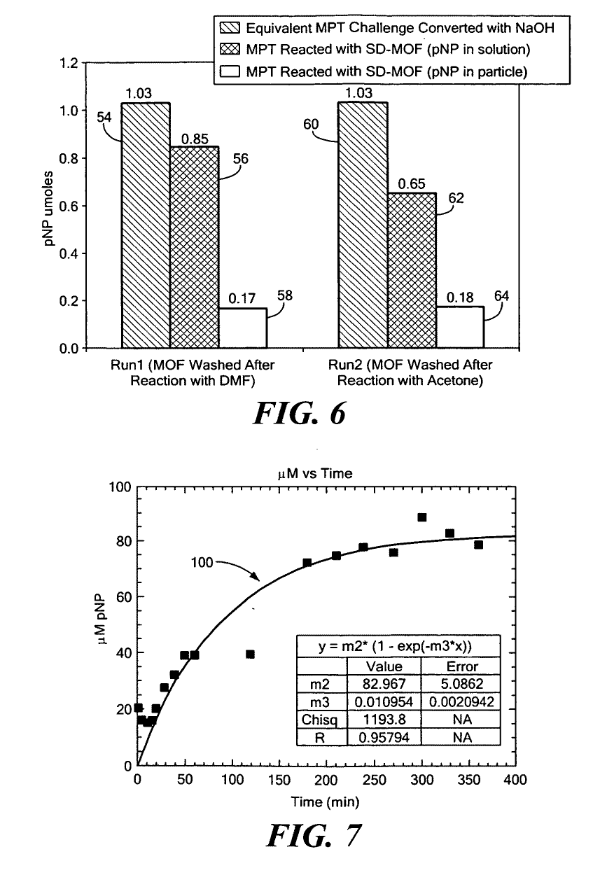

The kinetics of the MPT hydrolysis were then collected. Without the determining enzyme (OPH) incorporated, decontamination by the SD-MOF is complete within about 3 hours, much faster than any known catalytic particles, and hypersorptive for safe disposal. Out of 100 μM MPT, about 20% MPT or pNP was adsorbed to powders. Graph 100, FIG. 7 shows one example of SD-MOF of this invention decontaminating the MPT in a 15% methanol aqueous solution. In this example, the concentration of the degradation by-product pNP in solution was measured. As shown, an 80% bulk solution of pNP was achieved in about 300 minutes. The difference between the observed pNP concentration in the bulk solution and the expected 100 μM pNP can be attributed to sorption of pNP to the SD-MOF particles. Each reaction used about 100 mg of SD-MOF compound per 100 μmolar MPT.

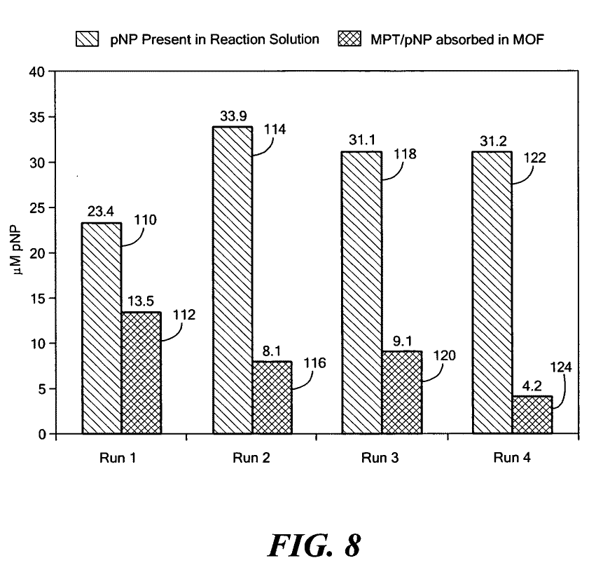

The SD-MOF of this invention can be reused many times. FIG. 8 shows one example where SD-MOF was reused four times, as shown by Run 1, Run 2, Run 3, and Run 4, indicated at 102, 104, 106, 108, respectively. In this example, the SD-MOF is rinsed with acetone between the runs and exposed to fresh MPT toxin. Each run was conducted for about 30 minutes. Graph 110 shows the pNP present in the reaction solution and Graph 112 shows the pNP sorbed to the particles of SD-MOF after rinsing with acetone. Similarly, graphs 114, 118 and 122 show the pNP bulk solution for Runs 2, 3, and 4, respectively and Graphs 116, 120 and 124 show the pNP particles sorbed by the SD-MOF after rinsing. As shown, the SD-MOF of this invention is able to effectively decontaminate the MPT and be reused many times. Each reaction used 100 mg of self-decontaminating metal organic framework per 100 μmolar MPT.

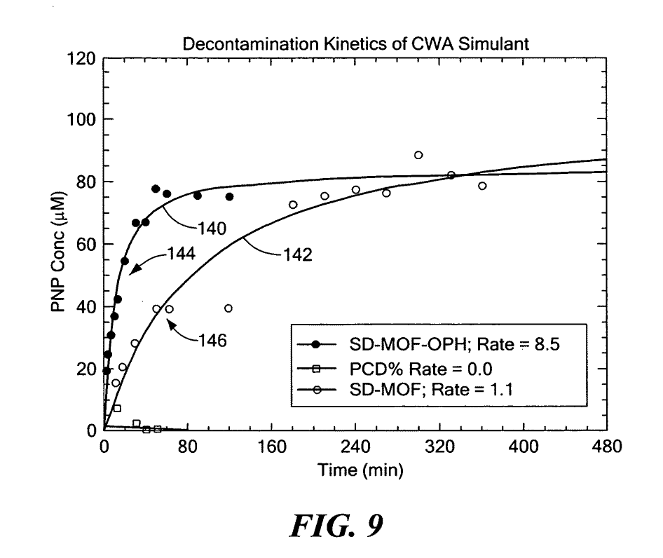

The SD-MOF of this invention can be used to support enzymes, such as OPH, to substantially increase its activity. Graph 140, FIG. 9, shows one example of the degradation of MPT to pNP by the SD-MOF of this invention coated with OPH. Graph 142 shows the degradation of MPT to pNP using SD-MOF without the OPH enzyme coating. As shown at 144 and 146, the OPH enzyme enhances the activity of the SD-MOF when compared to the non-coated SD-MOF. Each reaction used 100 mg of reactive adsorbent per 10 mL MPT (100 μmolar). Sorption of simulant by SD-MOFs is close to 20% while decontaminating 80% MPT out of 100 μM MPT in the solution. However, in case of Pyrazine based SD-MOF is not much absorptive, mostly decontaminating only.

Gas phase reactivity of the SD-MOF was also observed. Significant quantities of pNP were able to be extracted from SD-MOF powder sample after 24 hour exposure to methyl paraoxon (MPO) in a gas stream with no moisture. The amount of MPT/pNP produced were varied depending on the experimental conditions.

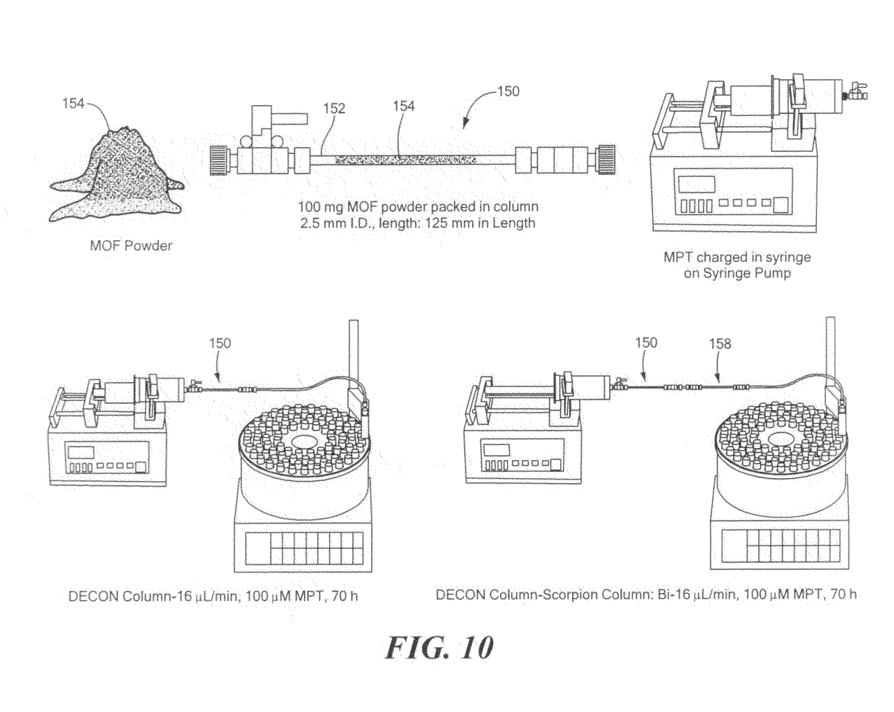

Continuous decontamination of MPT was demonstrated at a flow rate of about 1 mL/h in the SD-MOF powders of packed bed reactor 150 (PBR), FIG. 10. Column 152 was packed with SD-MOF powders 154, shown at 156, which continues to release out degraded breakdown product, pNP. To build complete/compact decontamination system, hypersorptive MOF-505 was filled in second column 158 connected to SD-MOF column 150 as a safeguard to sequester less toxic pNP much more for safe disposal. MOF-505 is a sorptive and non-reactive MOF which collects the degraded by-products produced by SD-MOF.

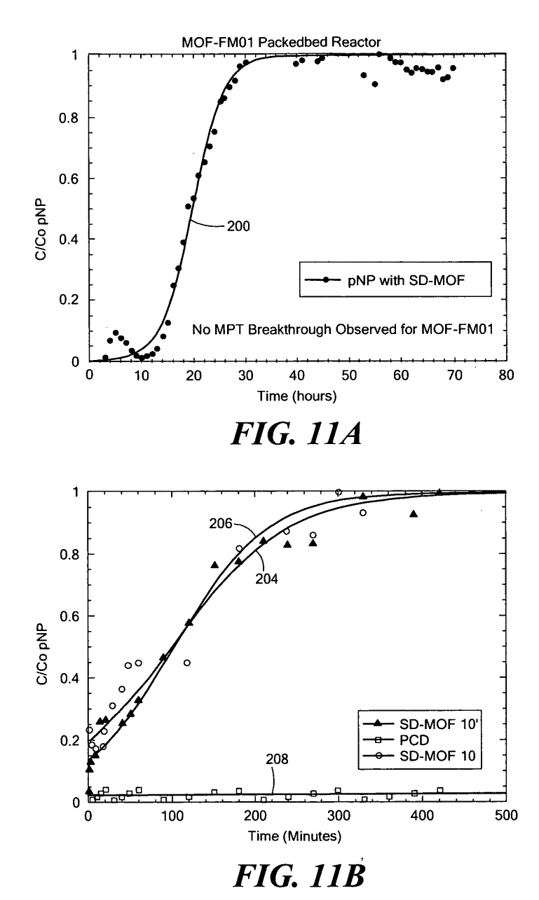

The observed activity from PBR 150, FIG. 10, filled with SD-MOF is shown by graph 200, FIG. 11A. Graph 200 indicates the breakthrough of the degradation by-product pNP was delayed for approximately 12 hours, indicated at 202. This means SD-MOF can effectively provide protection against CWAs and TICs, such as MPT for at least that amount of time.

FIG. 11B shows MPT degradation kinetics of SD MOF 10, FIG. 1A and SD-MOF 10′, FIG. 1B of this invention. MPTs degraded to pNP appeared in solution over a period of 8 h time period. PCD was a non-reactive adsorbent control. As shown by graph 204 for SD-MOF 10′, graph 206 for SD-MOF 10 and graph 208 for PCD, SD-MOF 10′ and SD-MOF 10 of this invention demonstrated the breakthrough of the by-product pNP released from the decontaminated MPT over the 8 hour time period.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

Other embodiments will occur to those skilled in the art and are within the following claims.

What is claimed is:

1. A self-decontaminating metal organic framework comprising:
an acid linked to a metal producing a metal organic framework configured for the sorption of chemical warfare agents and/or toxic industrial chemicals, the metal organic framework including reactive sites for the degradation of said agents and chemicals.

2. The system of claim 1 in which the acid is a triple bonded acid.

3. The system of claim 1 in which the acid is acetylenedicarboxylic acid (ADA).

4. The system of claim 1 in which the metal is copper nitrate.

5. The system of claim 1 in which the self-decontaminating metal organic framework is linked to the metal with a linking agent.

6. The system of claim 5 in which the linking agent includes Pyrazine, 2,6-dimethylpyrazine, 2-6-dichloropyrazine, dipyridylethlene, 4,4′-dipyridyl, or 2,3,5,6-tetramethylpyrazine.

7. The system of claim 1 further including an enzyme added to the metal organic framework to assist in the degradation of said agents and chemicals

8. The system of claim 1 further including a non-self-decontaminating metal organic framework added to the self-decontaminating metal organic framework.

9. The system of claim 1 in which the size of the pores of the self-decontaminating metal organic framework is tailored for specific said agents and chemicals.

10. The system of claim 1 in which the surface area of the self-decontaminating metal organic framework is tailored for specific said agents and chemicals.

11. A method for producing a self-decontaminating metal organic framework, the method comprising:
combining an acid with a linking agent and a metal to produce a self-decontaminating metal organic framework for sorption of chemical warfare agents and/or toxic industrial chemicals, the self-decontaminating metal organic framework including reactive sites for the degradation of said agents and chemicals.

12. The method of claim 11 in which the acid is a triple bonded acid.

13. The method of claim 11 in which the acid is acetylenedicareoxylic acid (ADA).

14. The method of claim 11 in which the metal is copper nitrate.

15. The method of claim 11 in which the linking agent includes Pyrazine, 2,6-dimethylpyrazine, 2-6-dichloropyrazine, dipyridylethlene, 4,4′-dipyridyl, or 2,3,5,6-tetramethylpyrazine.

16. The method of claim 11 further including the step of adding an enzyme to the metal organic framework to assist in the degradation of said agents and chemical.

17. The method of claim 11 in which the size of the pores of the self-decontaminating metal organic framework are tailored for specific said agents and chemicals.

18. The method of claim 11 in which the surface area of the self-decontaminating metal organic framework is tailored for specific said agents and chemicals.

19. A method of absorbing and degrading chemical warfare agents and toxic industrial chemicals, the method comprising:
adding a self-decontaminating metal organic framework to fabric or filter material, the self-decontaminating metal organic framework comprising an acid linked to a metal-organic framework configured for the sorption of chemical warfare agents and/or toxic industrial chemicals, the metal organic framework including reactive sites for the degradation of said agents and chemicals.

20. The method of claim 19 in which the acid is a triple bonded acid.

21. The method of claim 20 which the acid is acetylenedicareoxylic acid.

22. The method of claim 20 in which the metal is copper nitrate.

23. The method of claim 19 in which the self-decontaminating metal organic framework is linked to the metal with a linking agent.

24. The method of claim 19 in which the linking agent includes Pyrazine, 2,6-dimethylpyrazine, 2-6-dichloropyrazine, dipyridylethlene, 4,4′-dipyridyl, or 2,3,5,6-tetramethylpyrazine.

25. The method of claim 19 further including an enzyme added to the metal organic framework to assist in the degradation of said agents and chemicals.

26. The method of claim 19 in which the size of the pores of the self-decontaminating metal organic framework is tailored for specific said agents and chemicals.

27. The system of claim 19 in which the surface area of the self-decontaminating metal organic framework is tailored for specific said agents and chemicals.
